(19) United States
(12) Patent Application Publication
(10) Pub. No.: US 2010/0317094 A1
(43) Pub. Date: Dec. 16, 2010

(54) MILK ANALYSIS MICROFLUIDIC APPARATUS FOR DETECTING MASTITIS IN A MILK SAMPLE

(75) Inventors: Antonio Ricco, Los Gatos, CA (US); Jose L. Garcia Cordero, Dublin (IE); Richard O'Kennedy, Dublin (IE)

Correspondence Address: SEED INTELLECTUAL PROPERTY LAW GROUP PLLC, 701 FIFTH AVE, SUITE 5400, SEATTLE, WA, 98104, US

(73) Assignee: DUBLIN CITY UNIVERSITY, Dublin (IE)

(21) Appl. No.: 12/866,231

(22) PCT Filed: Feb. 4, 2009
(86) PCT No.: PCT/EP09/51284

§ 371 Date: Aug. 4, 2010

(30) Foreign Application Priority Data
Feb. 4, 2008 (GB) 0801991.1

(51) Int. Cl. C12M 1/34

(52) U.S. Cl. 435/287.2; 435/287.1; 435/287.3; 435/288.7

(57) ABSTRACT

A milk analysis apparatus for detecting mastitis in a milk sample by isolating somatic cells in the form of a pellet using centrifugal sedimentation is described. The apparatus comprises a vessel for holding the milk sample, and a centrifuge for rotating the vessel. The vessel includes an inlet for facilitating charging milk into a body portion of the vessel; and a trap for capturing somatic cells suspended in the milk sample. The somatic cells are biased towards the trap upon rotation of the vessel.

### FIELD OF THE INVENTION

The present invention relates to a milk analysis apparatus. In particular the invention relates to a microfluidic apparatus for detecting mastitis in a milk sample by isolating somatic cells suspended within the milk sample by using centrifugal sedimentation to form pellets composed of the somatic cells. The invention further relates to a cytometer incorporating such a milk analysis apparatus. The invention further relates to a milk analysis apparatus for detecting mastitis in a milk sample and which in addition to an analysis based on centrifugal sedimentation provides at least one other test of the milk sample quality selected from: a determination of the quantity of cream or fat within the milk sample; a determination of the concentration of protein within the sample; and/or an enzymatic analysis of the milk sample.

### BACKGROUND

Liquid analysis typically involves the determination of the presence of or relative volume of one of a number of constituents within a test sample of the liquid. The specifics of the analysis, for example, what is the constituent being searched for, will of course depend on the nature of the liquid analysis being conducted.

For example, bovine mastitis (BM) affects the composition of milk by altering the concentration of certain proteins, enzymes, fat, and ions, and also by increasing the number of somatic cells in milk. It is well known that the number of somatic cells dramatically increase after a pathogen invades the teats and/or udder of a cow. Somatic cells are a set of mainly white blood cells and epithelial cells. Determining the number of somatic cells present in milk has become the standard in diagnosing early signs of mastitis and is also used to estimate the monetary and qualitative value of the milk. After years of research, guides have been established that define a threshold of 200,000 cells per ml as an inflection point to determine if a pathogen has invaded a teat of a cow. Generally, a cow sheds 50,000 to 200,000 somatic cells per ml in milk. If a cow sheds a number of somatic cells greater than the threshold it indicates that the animal is trying to fight an infection, resulting in more white blood cells being present in the milk.

Most commercial assays that count the number of cells in a solution involve the tagging of cells with a fluorophore. Once tagged, cells can then be detected, typically in one of two ways. A cytometer may be used for counting the tagged cells one at a time. Alternatively, cells may be counted using a microscope and image processing software. Although these techniques are very sensitive and accurate, they require reagents and dyes to label the cells and a detection system equipped with advanced optics. The primary reason why the cells are tagged is that the cells are intermingled with other constituents and need to be distinguished from the other constituents. It will be understood that such techniques typically require laboratory analysis and as such cannot be done locally on a farm where the cows are located.

There is therefore a need for a milk analysis apparatus which addresses at least some of the drawbacks of the prior art.

These and other features will be better understood with reference to the following Figures which are provided to assist in an understanding of the teaching of the invention.

### SUMMARY

These and other problems are addressed by provision of a milk analysis apparatus for detecting mastitis in a milk sample by isolating somatic cells suspended therein using centrifugal sedimentation.

Accordingly, a first embodiment of the invention provides a milk analysis apparatus as detailed in claim 1. Advantageous embodiments are provided in the dependent claims. The invention also provides a method as detailed in claim 57. Advantageous embodiments are provided in the dependent claims. Additionally, the invention relates to a milking system as detailed in claim 64. Furthermore, the invention relates to a cytometer as detailed in claim 65. A multi-parameter milk analyser as claimed in claim 66 is also provided.

These and other features will be better understood with reference to the followings Figures which are provided to assist in an understanding of the teaching of the invention.

### BRIEF DESCRIPTION OF THE DRAWINGS

The present invention will now be described with reference to the accompanying drawings in which:

FIG. 1 is a perspective view of a vessel assembly of a milk analysis apparatus of FIG. 6.

FIG. 2 is a perspective view of a vessel of the vessel assembly of FIG. 1.

FIG. 3A is an isometric perspective view of the vessel of FIG. 2.

FIG. 3B is a plan view of the vessel of FIG. 3A.

FIG. 3C is a view from beneath the vessel of FIG. 3A.

FIG. 3D is an exploded plan view of a detail of the vessel of FIG. 3A.

FIG. 4 is a cross sectional view of the vessel along the line I-I′.

FIG. 5 is a cross sectional view of the vessel along the line A-A′ of FIG. 4.

FIG. 6 is a perspective view of a milk analysis apparatus in accordance with the present invention.

FIG. 7 is a side view of the blind channel of five vessels.

FIG. 8 is cross sectional side view of the vessel of FIG. 3A.

FIG. 9 is a diagrammatic view of a sphere in a stream of liquid.

FIG. 10 is a diagrammatic free-body diagram of a sphere.

FIG. 11 is a side exploded view of a portion of the vessel of FIG. 2.

FIG. 12 is a perspective view of a plastic injection mould used to mould the vessel of FIG. 2.

FIG. 13 is a perspective view of the vessel of FIG. 2.

FIG. 14 is graph illustrating cell count versus area.

FIG. 15 is graph illustrating fat percentage versus length.

FIG. 16 is a diagrammatic view of a photodetection arrangement which may be used in conjunction with the vessel assembly of FIG. 1.

FIG. 17 is another vessel assembly.

FIG. 18 is a plan view of one of the vessels of the vessel assembly of FIG. 17.

FIG. 19 is an exploded view of a detail of the vessel of FIG. 18.

FIG. 20 is a perspective view of a plastic injection mould used to mould the vessel of FIG. 18.

FIG. 21 is a cross sectional view of the vessel of FIG. 18.

FIG. 22 is a plan view of the vessel of FIG. 18 including dimensions in millimeters.

### DETAILED DESCRIPTION OF THE DRAWINGS

The invention will now be described with reference to some exemplary milk analysis apparatus which are provided to assist in an understanding of the teaching of the invention. While the milk analysis apparatus are described with reference to a quantitative analysis of a sample milk volume to determine the presence or otherwise of somatic cells, it will be understood that the milk analysis apparatus provided in accordance with the teaching of the present invention could be used in other types of milk analysis such as estimating the fat content in a milk sample.

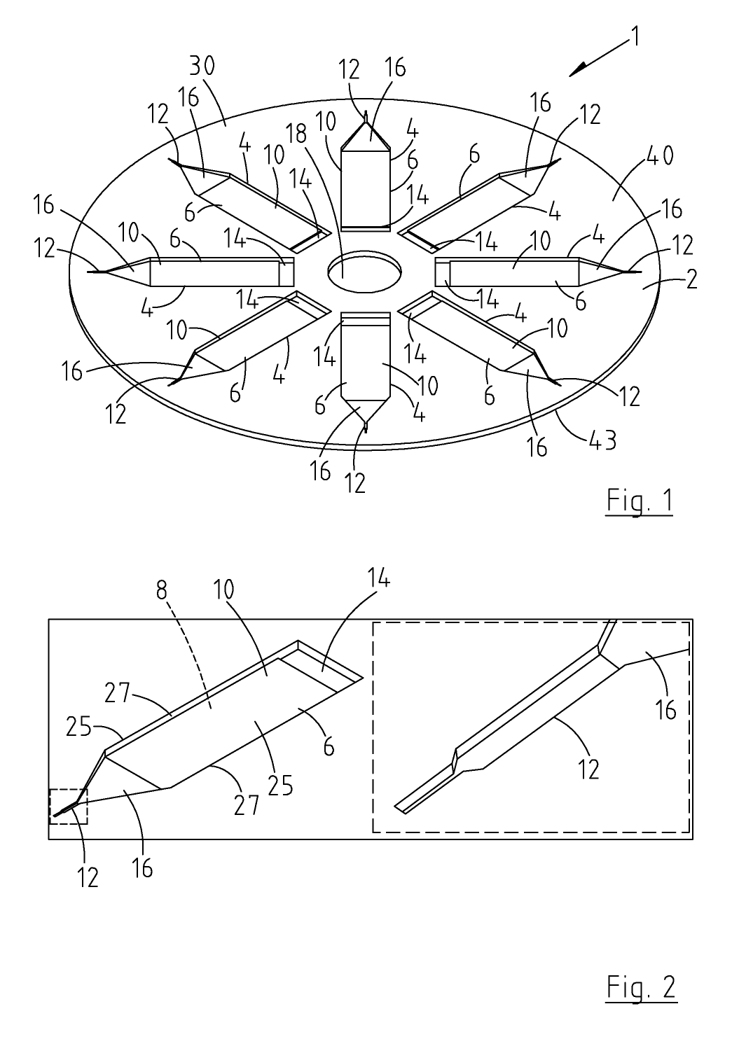

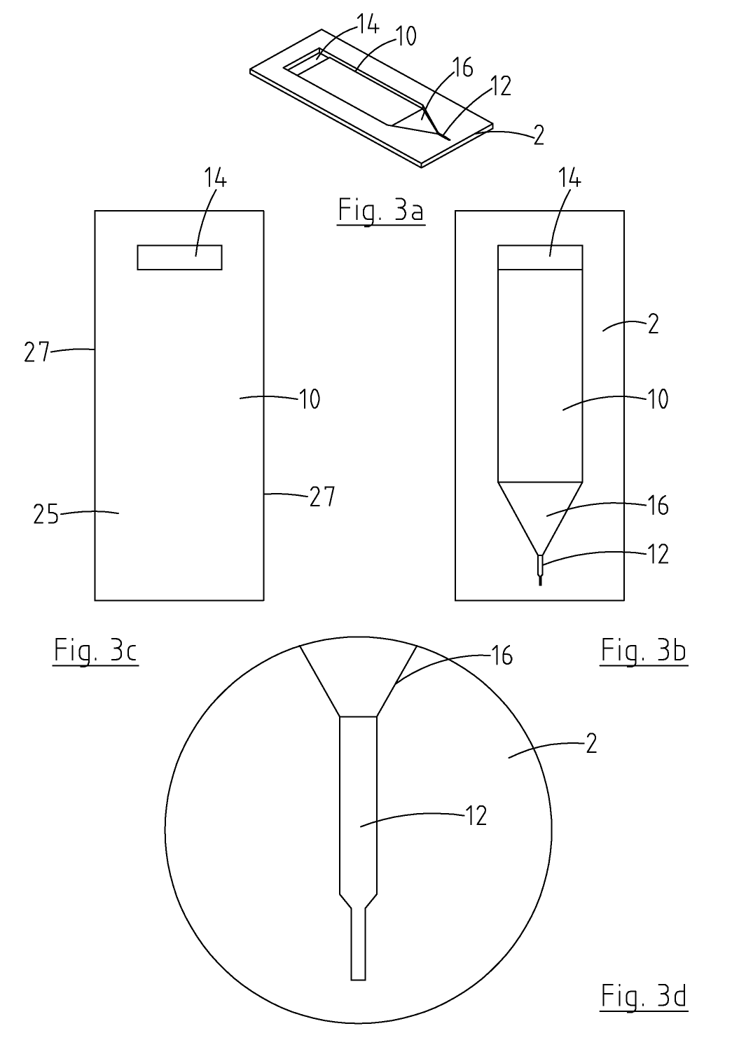

Referring to the drawings and initially to FIG. 1 there is provided a vessel assembly 1 of a milk analysis apparatus which holds a plurality of milk samples during analysis thereof. The vessel assembly 1 comprises a carrier member in form of a plastic disc 2 with dimensions substantially corresponding to that of a conventional compact disc (CD). The disc 2 supports optical transmission of light therethrough for facilitating optical analysis of the milk samples. A plurality of vessels 4 are formed on the disc 2 at radially spaced apart locations, each of the vessels 4 are designated for holding a corresponding milk sample of an associated teat. In this exemplary arrangement, the vessels 4 are provided equidistant apart and extend radially from a central opening or mid-point 18 of the CD. In this way they are circumferentially arranged about the mid point.

Each of the vessels 4 comprises a main body 6 defining a hollow interior region 8 for accommodating the milk samples therein. Each main body 6 includes an elongated body portion 10 defining a main volume of the vessel 4 in fluid communication with an elongated trap 12 defining a minor volume of the vessel 4. The trap 12 has a transverse cross sectional area which is substantially less than that of the body portion 10 thereby defining a constricted region for accommodating and/or capturing predetermined constituents, such as for example, somatic cells which are in the form of cells suspended in the milk sample. Inlets 14 are formed on the body portions 10 through which the milk samples are loaded to the hollow interior regions 8. The traps 12 are distally located relative to the inlets 14, and are also distally provided to the mid-point 18 of the disc 2 such that a centrifugal force resultant from rotation of the disc will bias the somatic cells towards the traps. A guide portion in the form of a tapered V-shaped funnel 16 is located intermediate each body portion 10 and corresponding trap 12 for guiding the somatic cells from the body portion 10 into the trap 12.

The central opening 18 is formed on the disc 2 for accommodating a rotatable spindle 20 of a centrifuge, in this exemplary arrangement, provided in the form of a disc drive unit 22 which rotates or drives the disc 2 as illustrated in FIG. 6. It will be appreciated that within the context of the present teaching that any means for effecting a rotation of the disc to effect a separation of constituents of the fluid by centrifugal sedimentation may be considered a centrifuge and the drive unit of a CD or DVD player is a particular embodiment of same. In this context, the disc drive unit 22 could be of the form substantially similar to the type of disc drive units which read CDs and such an arrangement will be familiar to any person knowledgeable in the operation of compact discs drives. The longitudinal axes 23 of the vessels 4 extend radially from the spindle 20 such that the vessels 4 define corresponding equally spaced apart spokes on the disc 2. In this way they can be considered as being radially distributed about the disc. As was mentioned above each of the vessels 4 terminate in corresponding traps 12 which are distally located from the spindle 20 whereas the body portions 10 are located proximal to the spindle 20. In an exemplary arrangement the body portions 10 define a pair of spaced apart major surfaces 25 with a pair of spaced apart minor surfaces 27 extending therebetween. The inlets 14 are desirably formed on the major surfaces 25 and are available from an upper major face 30 of the disc 2 such that a fluid would be introduced downwardly into the body portions 10 in a direction substantially transverse to the longitudinal axis 23 of the vessels 4. The traps 12 are defined by corresponding blind channels which are in fluid communication with the body portions 10. The disc drive unit 22 rotates the disc 2 thereby generating a centrifugal force which biases or urges the somatic cells from the body portions 10 into the traps 12.

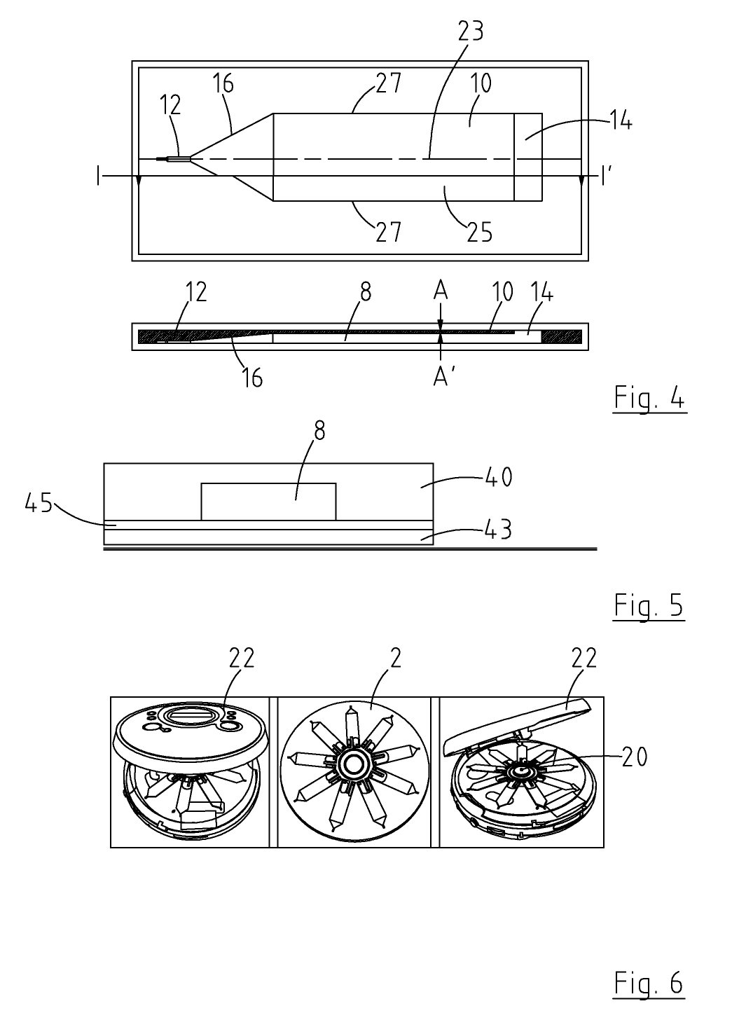

The disc 2 may be fabricated from any suitable material and in a first arrangement comprises a circular polymer substrate sheet 40 of cyclo olefin copolymer bonded to a circular polymer cover sheet 43 of polymethy methacrylate by a pressure adhesive layer 45, as illustrated in FIG. 5. It will be appreciated by those skilled in the art that the substrate sheet 40 and the cover sheet 43 may however be of any suitable polymer, and it is not intended to limit the invention to cyclo olefin copolymer or polymethy methacrylate. Alternatively, the substrate sheet 40 may be bonded to the cover sheet 43 by thermal lamination or other suitable processes without the aid of the pressure adhesive layer 45. In the example of FIG. 1 the vessels 4 are integrally formed in the disc 2, each of the vessels 4 are formed by milling recesses into the substrate sheet 40 with a milling machine (not shown). Alternatively, the vessels 4 may be formed using conventional moulding and extrusion processes or other micro-fabrication techniques know in the art. The milled or moulded plastic substrate sheets 40 are then bonded to the cover sheet 43 for support. The substrate sheet 40 and the cover sheet 43 form respective opposite major faces of the disc 2.

The vessel assembly 1 is particularly suitable for isolating somatic cells in a milk sample by centrifugal sedimentation so that the isolated somatic cells can be easily identified or even counted to determine if the milk sample is contaminated with mastitis without for example the need to tag the somatic cells with a fluorophore. By forcing the somatic cells into the blind channels defined by the traps 12 it is possible to separate or isolate these cells from other constituents of the milk which will be provided within the body portions 10. It will be understood that milk comprises a plurality of various constituents and is composed in its majority of water, fat and proteins. Present in less quantities are vitamins, minerals, gases, and somatic cells. Somatic cells differ from the other constituents in milk mostly in size (ten times larger than bacteria and a thousand times larger than most proteins) but also in density. Somatic cells have a similar size to fat globules but have a greater density than fat globules and water. An apparatus provided in accordance with the present teaching is designed to take advantage of this dissimilar characteristics to first separate somatic cells based on their weight (sedimentation principle) and then to concentrate the cells into a constricted area to facilitate their enumeration. To achieve this separation within a reasonable time frame, the vessel assembly 1 is rotated by the disc drive unit 22 to speed up the process of separation. Given the low number of somatic cells present in milk, typically less than 0.01% of its volume, it will be appreciated that the amount of milk that each vessel 4 must be able to hold needs to be large enough to contain a representative number of somatic cells. The arrangement operates based on a preferential forcing of the somatic cells into the blind channels, the forcing being achieved based on the density differential between the somatic cells and the other constituents of the milk. As a result of this density difference, on rotation of the disc 2, the somatic cells are preferentially directed under the influence of centrifugal force resultant from rotation of the disc into the blind channel of the trap 12. On receipt therein the number of somatic cells can be counted thereby determining if a cow has mastitis. Alternatively the identification that a predefined volume of the trap is filled with cells may be sufficient to indicate the presence of mastitis without requiring an actual counting of the number of cells trapped within the trap. It will be understood that the presence of a predetermined number of cells relative to the test sample volume is indicative of the presence of mastitis, and it is the identification of such a ratio within a sample volume that provides an output from an apparatus provided in accordance with the present teaching. In this context it will be understood that a counting of the individual cells captured within the trap is not necessary in that once a plurality of such somatic cells are moved into the trap formed by the constricted region they form a pellet that can be optically identified. The final dimensions of the pellet will be dependent on the number of somatic cells within the milk volume in accordance with the present teaching. Identification of a pellet of a particular size may be used to ascertain the presence of mastitis within the sample volume.

While the vessel assembly 1 has been described providing a component of a milk analysis apparatus, it will be understood that the vessel assembly 1 may be used during analysis of any liquid whose constituents may be differentiated based on their relative densities.

In the exemplary arrangement shown in FIG. 1, in use, milk samples are obtained from eight different cows which are then loaded to corresponding ones of the vessels 4 through the inlets 14. The vessels 4 maybe manually loaded with milk using a pipette. Alternatively, the vessel assembly 1 can be integrated with a milking system. Milking systems comprise a plurality of discrete milking units so that a number of cows can be milked simultaneously. Typically each milking unit consists of four cups with pneumatic liners located therein for accommodating corresponding teats of the cow for extracting the milk from the udder of the cow. The cups are in fluid communication with a holding jar which holds the milk as the cow is being milked. Once the cow is completely milked the milk in the holding jar is pumped via a cooling system to a refrigerated central tank. In the event that the cow has bovine mastitis and the infected milk is pumped from the holding jar to the central tank all the milk in the central tank becomes contaminated thereby reducing the monetary value of the milk. Prior to pumping the milk from the holding jar to the central tank a sample of the milk from the holding jar is delivered to the vessel assembly 1 so that the number of somatic cells in the milk sample can be determined. If the sample of milk taken from the holding jar contains a number of somatic cells greater than the threshold level of 200,000 cells per ml it indicates that the animal is trying to fight an infection by recruiting more white blood cells and may have bovine mastitis. In this scenario, rather than risking contaminating the rest of the milk contained in the central tank the milk in the holding jar is disposed of.

If one of the vessels 4 containing a milk sample was left standing still as a result of the influence of the earth's gravitational field, the somatic cells/cells would, over a period of time, fall from the body portion 10 to the bottom of the V-shaped funnel 16 as they have the greatest density. The fat globules would form a cream band towards the inlet 14 in the body portion 10 as the fat globules have the next greatest density. It is expected that over time the somatic cells/cells would then fall from the V-shaped funnel 16 to the trap 12 and pack, although poorly, in the blind channel of the trap 12. The teaching of the invention provides for the use of centrifugal forces derived from a centrifuge to accelerate this process and to bias the separation of the milk into its components of different densities.

The disc 2 containing the milk samples is loaded to the disc accommodating area of the disc drive unit 22 such that the spindle 20 of the disc drive unit 22 extends through the central opening 18. The disc drive unit 22 is operated for revolving the disc 2 in a similar manner that the disc drive unit 22 would rotate a compact disc. Centrifugal forces resulting from rotating the disc 2 urge the somatic cells/cells into the blind channels 35 of the traps 12 while the fat globules settle in a region at the top of the sample towards the inlet 14 thereby forming a band in the body portion 10. The purpose of the blind channel 35 is to allow the packing of the somatic cells/cells to fill the trap for example in the form of columns which increase in size proportionately to the number of somatic cells present in the milk sample. If such a regular packing is achieved (although it will be appreciated that such a regular form is not essential for the identification of somatic cells within the trap) the columns formed by the somatic cells define a matrix which results in the somatic cells having a uniform distribution so that they may be counted as a cluster rather than one by one, as it is done by most commercial devices.

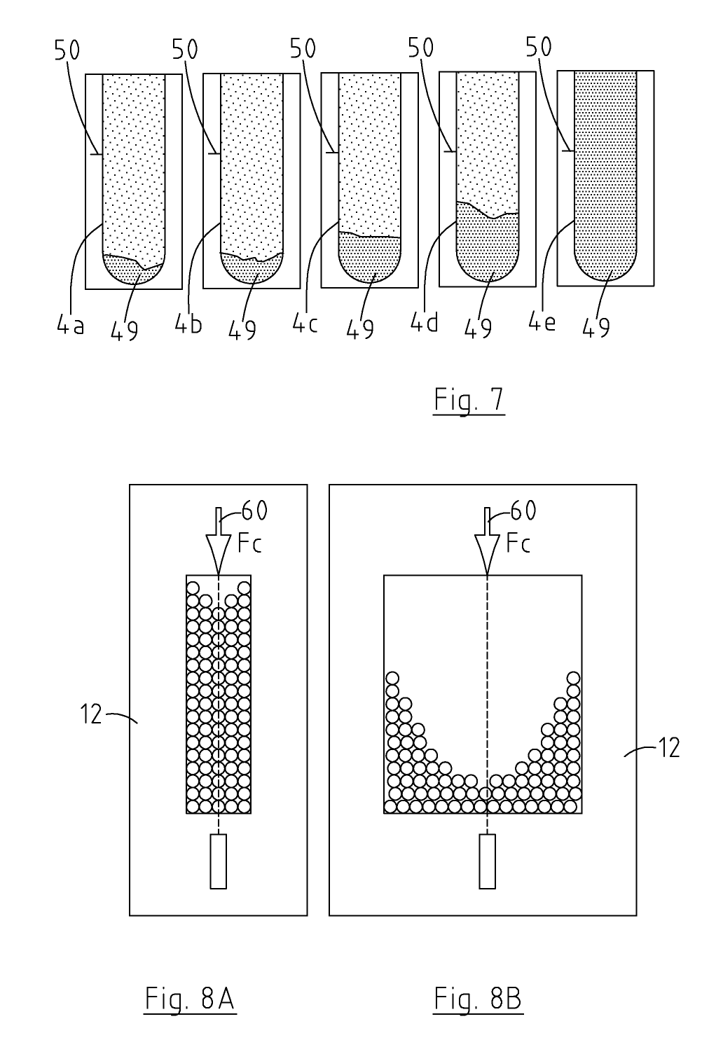

Heretofore, it will be appreciated that the arrangement takes advantage of the rotating spindle provided as part of a conventional disc drive unit. In a modification or further embodiment, the optical head of the disc drive unit 22 can be used for providing an optical analysis of the sample volume for processing purposes. Referring now to FIG. 7, the milk samples of five vessels 4a, 4b, 4c, 4d and 4e were photographed to enable an image analysis of the vessels. In these images, the dark shaded areas 49 indicate the presence of somatic cells. It will be seen that the dark shaded areas 49 of the vessels progressively increase from the vessel 4a to the vessel 4e. By providing suitable image processing software it is possible to provide a threshold indicia 50 on the photograph which substantially corresponds to the threshold level of 200,000 cells per ml. Alternatively, the threshold indicia 50 could be marked on the trap 12. If the shaded area 49 is above the threshold indicia 50 it indicates that the cow has bovine mastitis. It will be seen from a comparison of each of the vessels in FIG. 7 that the dark shaded area 49 in vessel 4e exceeds the threshold indicia 50. It will therefore be appreciated that this is indicative that the cow which provided this sample has mastitis. This cow's milk is disposed of rather than being pumped to the central tank where it would contaminate the rest of the milk in the central tank. It will be understood that using an arrangement provided in accordance with the teaching of the invention that it is possible to conduct this analysis at the point of milking without requiring a transport of the collected milk samples to a laboratory for analysis by a third party. Thus a person with no or little scientific skills such as a farmer may conduct the analysis of a milk sample to determine if a cow has mastitis locally on the farm. The milk analysis apparatus may also include a means for quantifying the number of somatic cells in the trap 12, and also a means for quantifying the fat content of the milk sample by measuring the characteristics of the band of fat globules. The quantifying means may comprise for example, a linear laser diode provided as a component of the optics of the disc drive unit 22, or a capacitive or impedance sensor. The quantifying means which are described above are given by way of example only and are not intended to be an exhaustive list. Alternative quantifying means may be used which would be readily apparent to a person skilled in the art in the context of the present invention.

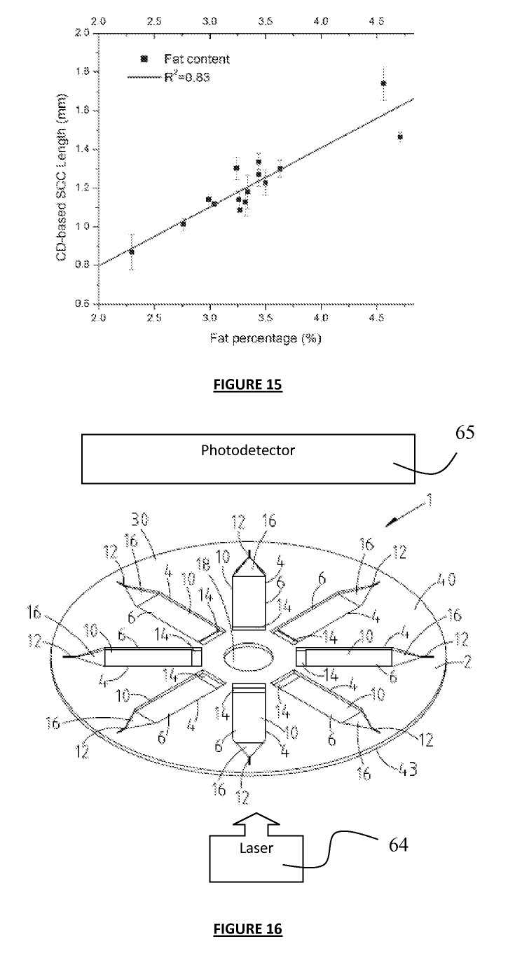

It will be appreciated that heretofore the use of an apparatus in accordance with the present teaching has been described with reference to identification or somatic cells within a sample milk volume. As was discussed above the separation of the milk sample volume into its constituents based on density allows for this identification of the somatic cells but may also serve to separate the other constituents of the milk which may also be identified. As mentioned above, the separation may serve to generate a band of identifiable fat above the somatic cells that are located within the trap. Those skilled in the art of dairy processing will appreciate that fat percentage information on individual animals is valuable for determining the commercial value of the milk produced by each cow in a herd. Milk fat content is one factor which sets the milk price paid to the farmer by dairies. Milk fat content information also allows a farmer to evaluate different nutrition programs for groups of cows to determine the most effective. In contrast, central tank milk component information is a weighted average of all cows in the herd, limiting its usefulness in evaluating any one particular individual or group of animals. In a similar fashion to that described with reference to the somatic cells, the rotation of the vessels with the milk volume provided therein will effect generation of an identifiable fat band within the vessels. Typically the fat globules will settle in a region toward the inlet 14 in the body portion 10 and the concentration of fat may be estimated by measuring the length of this fat band. FIG. 15 shows a graph illustrating fat percentage versus the length of the fat band. Using an optical analysis similar to that described with reference to the somatic cell identification, an apparatus provided in accordance with the present teaching may be used to provide an extremely fast and cost-effective method of estimating milk fat content. Thus a person with no or little scientific skills such as a farmer may estimate the fat content of milk simply by measuring the length of the fat band. Indicia may be provided on each vessel 4 of the vessel assembly 1 for facilitating fat content measurements. Analytic software may be used for estimating the fat content based on the length of the fat band. The optical head of the disc drive unit 22 can be used for identification of the fat band. The identification of the fat band can be effected in parallel with or independently of the identification of the somatic cells.

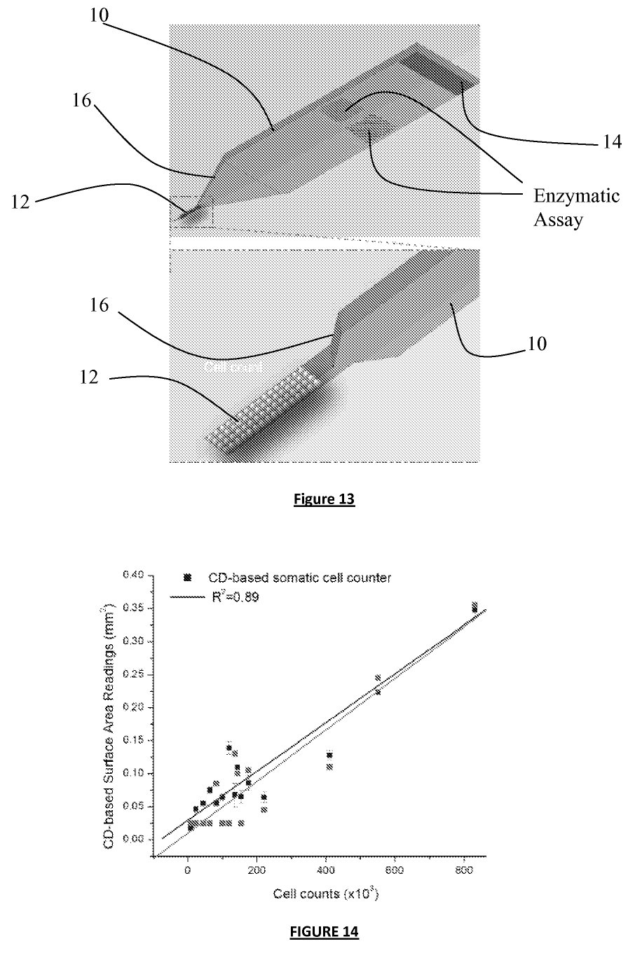

As was mentioned above, it will be understood that the somatic cells do not need to be counted individually for determining if the cow has bovine mastitis as they can be counted in clusters. The use of the vessel assembly 1 therefore simplifies the detection mechanism and the time needed to read the results. The blind channel of each vessel 4 has to be of sufficient length to accommodate the maximum number of cells from a sample of a cow suffering a chronic condition. If the blind channel is too wide, the centrifugal vector force would be greatest at the centre of the blind channel and less pronounced away from the centre resulting in a non-uniform distribution of the somatic cells, which would complicate the detection and measurement of the somatic cells. The blind channel of FIG. 8A is correctly dimensioned such that the cells have a uniform distribution. However, the blind channel of FIG. 8B is too wide resulting in the cells arranging in a non-uniform fashion. The blind channel needs to be narrow and shallow enough to give a quantitative result if an optical detection system 60 is based upon scanning the size of the cluster along the length of the middle-section of the blind channel. Nevertheless, it will be appreciated by those skilled in the art that an impedance or capacitance reading system, using embedded electrodes in the device either in direct or external contact with the solution, would be able to resolve the number of cells, no matter what the dimensions of the blind channel were. It will therefore be appreciated that the important features of the blind channel are that it selectively captures milk constituents, in this case somatic cells, of a predetermined relative density to those of other constituents within the milk sample, the geometry required to do so being less critical. FIG. 14 shows a graph of cell count versus the area of the blind channel. A counter using photographic analysis may be used for counting the number of somatic cells in the blind channel.

To achieve proper analysis thresholds it is useful to have a determination of the maximum and minimum number of cells that are expected from the samples. In this exemplary arrangement of milk analysis, it is well acknowledged within the art that a range of 50,000-200,000/ml cells are present in milk for a healthy animal whereas for an unhealthy animal, milk can contain up to 3,000,000 cells/ml. These limits, 50,000 and 3,000,000 cells/ml, will be appreciated are useful in setting the first constraint on the dimensions of the blind channel of the trap 12.

It is also necessary to know the type of the cells present in the liquid sample (be that milk or any other liquid type) and their characteristics, mainly volume and density. Somatic cells in milk encompass four different types of cells, namely, neutrophils, macrophages (a type of monocyte), lymphocytes, and epithelial cells. Depending on the condition of the animal, cells would appear in milk in different proportion as shown from the table below. Macrophages and neutrophils form the highest concentration of the cells in milk as shown in Table 1.

TABLE 1
Proportion of cells present in found in normal and infected milk

| | | Sub-clinical |
|---|---|---|
| Cell Type | Normal Milk | mastitis |
| Neutrophil | 0-11% | >90% |
| Macrophage | 66-88% | 2-10% |
| Lymphocyte | 10-27% | 2-10% |
| Epithelial cells | 0-7% | 0-7% |

As illustration of the possible use of a system provided in accordance with the teaching of the present invention outside a bovine environment, the physical properties of human blood cells are listed in the table (2) below. Mammalian cells, in general, would have similar physical properties and it will be appreciated therefore that the vessel assembly 1 when driven by a centrifuge would also have application in such environments.

TABLE 2
Physical Properties of blood cells

| | Diameter | Surface | Volume | Mass |
|---|---|---|---|---|
| Cell type | Mm | area μm² | μm³ | density g/cm³ |
| Leukocytes (WBC) | 6-10 | 300-625 | 160-450 | 1.055-1.085 |
| Neutrophils | 8-8.6 | 422-511 | 268-333 | 1.075-1.085 |
| Eosinophils | 8-9 | 422-560 | 268-382 | 1.075-1.085 |
| Basophils | 7.7-8.5 | 391-500 | 239-321 | 1.075-1.085 |
| Lymphocytes | 6.8-7.3 | 300-372 | 161-207 | 1.055-1.070 |
| Monocytes | 9-9.5 | 534-624 | 382-449 | 1.055-1.070 |
| Erythrocytes (RBC) | 6-9 | 120-163 | 80-100 | 1.089-1.100 |
| Thrombocytes | 2-4 | 16-35 | 5-10 | 1.04-1.06 |

The average size of a somatic cell is in the range of 6 to 10 μm and has a volume spanning 160 to 450 μm³. It is thus reasonable to assume that somatic cells have an average size of 8 μm and a volume of 165 μm³.

The characteristics and performance of the sensor would dictate the minimum dimensions of the blind channel. It will be appreciated however that the optical arrangement of conventional disc drive units 22 of a CD player can be usefully employed in imaging down to 2 μm.

Given that one of the advantages of the vessel assembly 1 of the milk analysis apparatus is that it is itself embedded in the footprint of a CD, it is useful to preserve as much as possible the original dimensions and weight of the CD so as to exploit to the maximum the same technologies that make functional a CD, such as CD enclosures, accessories, motors, and optical detection systems. For example, all CD players have holders that can only fit discs of 1.2 mm thick, so CD's thicker than this dimensions would not be optimal and would require a special adaptor or a new holder. Nevertheless it will be appreciated while this is an optimal design characteristic, it is not intended to limit the application of the teaching of the present invention to any one set of geometrical parameters, be that the thickness of the apparatus or any other parameter.

Another consideration to take into account is the number of vessels 4 that could be desirable or likely to be contained on the disc 2. As it is desirable to test each teat of a cow simultaneously—typically 4 teats per animal—it is advantageous that the number of vessels provided on any one disc is a multiple of 4. Furthermore, it is desirable that the disc 2 remains substantially in stable equilibrium as it is rotated by the disc drive unit 22. An even number of vessels 4 with similar volumes are therefore provided which are equally spaced apart along the disc 2 so that the vessels 4 provide a uniform distribution of weight across the disc 2 as it rotates. The space is limited to the surface area of the disc 2, roughly 11100 mm², and to be consistent, the height of all vessels 4 must be less than 1.2 mm, as discussed above. Also, the maximum length of the vessels 4 is given by the radial dimensions of the disc 2 which is 53.5 mm, for an inner and outer radius of 7.5 and 60 mm, respectively. But practical and manufacture considerations would require about 5 mm of radial space from each side of the edges of the disc, setting this limit to about 43.5 mm. The radius of the inner and outer edge of the disc 2 then becomes 12.5 and 55 mm, respectively.

It is then evident that the more volume of a sample a vessel 4 holds the less number of vessels 4 a vessel assembly 1 can accommodate. The dimensions of the vessels 4 have to be chosen by first considering a sample volume as small as possible. Nevertheless, it is important to consider that the vessel assembly 1 may be used by a person with no pipette experience that would allow dispensing a minute and exact amount of milk into the vessel assembly 1a difficult task. Pipettes are expensive and require careful operation. If instead a dropper is used, a minimum quantity of about 150 μL could be dispensed more or less accurately.

If the device holds 150 μL of milk, the blind channel would have to accommodate up to 450,000 cells which corresponds to a maximum of three million cells per ml. The volume of such a number of cells is 0.074 μL. It is desirable to have a relative long blind channel so that the length of the column of cells increases proportionately to the number of cells in the sample. In order to deduce the width, height and total length of the blind channel it is necessary to select a predetermined increment in unit of length to correspond to a predetermined increment of the number of cells. For example, an increment of a 100 μm in length corresponds to an increment of 10,000 cells. The total volume of 10,000 cells is 1,650,000 μm³, which divided by the 100 μm desired increments would give 16,500 μm². The width and height of the channel can be found from this resulting area by calculating the square root, which finally give 130 μm³. The height of the blind channel can be deduced by assigning a predetermined value to the width, for example 200 μm and deducing the height therefrom. The total length of the blind channel can be found by dividing the total volume of the maximum number of cells, 0.074 μL, by the volume occupied by each 100 μm increment, 1650000 μm³, which gives a total length of 4.5 mm.

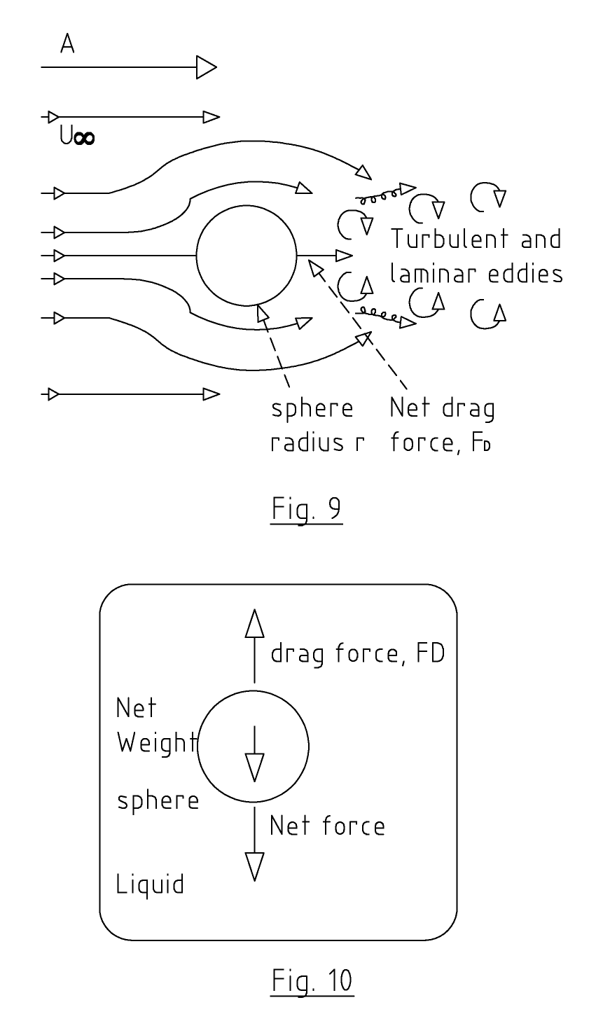

As illustrated in FIG. 9 the individual somatic cells in the blind channel are generally spherical in form. A sphere of radius r situated in a fluid stream under laminar conditions will be literally dragged by the encapsulating or surrounding fluid in which it is located. As illustrated in FIG. 9, the upstream velocity profile far away from the sphere is well defined, but upon hitting the sphere, turbulent eddies and laminar vortices will develop downstream from the sphere. These turbulences give rise to a pressure difference between the upstream and downstream sides of the sphere, impelling a net form drag on the sphere in the direction of the flow indicated by arrow A. In addition to these turbulences, velocity gradients develop near the sphere which impart a net viscous drag on the sphere in the direction of the flow. The mathematical expression that relates the net drag force due to these two effects is known as Stoke's law. Stoke's law is proportional to the velocity of the fluid, u_{∞}, and a frictional coefficient, f_{F}, which depends on the characteristics of the particle. For a sphere, Stoke's law can be expressed as:

F_{D}=6πμrₛu_{∞} (1)

### Where:

rₛ represents the radius of the sphere,
μ the viscosity of the medium, and
u_{∞} the velocity of the fluid.

Stoke's law also holds for a sphere moving in a still fluid. For this case the velocity of the fluid, u_{∞}, in equation (1) has to be replaced by the velocity of the particle moving upwards. If both, object and fluid, are displacing at distinct velocities, then the drag force is in the direction of the relative velocity u_{∞}−uₚ, and Stoke's law will still be valid under these circumstances.

When a sphere is settling under gravity in a liquid it will be observed that at first the sphere will accelerate but at the same time a drag force is created by the displacement of the sphere that tries to slow it down. Eventually, the drag force will counterbalance the net weight of the sphere and there will be no more acceleration, so that the sphere will fall with a constant terminal velocity, also called velocity of sedimentation.

Referring now to FIG. 10 which illustrates a free-body diagram of forces acting on a spherical particle. From the free-body diagram of FIG. 10 equation 2 can be deduced.

Net weight−Drag Force=Rate of increase of momentum

$${V}_{S} ( {ρ}_{S} - {ρ}_{F} ) g - {F}_{D} = {V}_{S} {ρ}_{S} \frac{d u}{d t} \qquad ( 2 )$$

### Where:

V_{S} is the volume of the sphere (which equals 4πr_{S}³/3),
ρ_{S} is the density of the sphere,
ρ_{F} the density of the medium,
g is gravity,
F_{D} is the drag force, and
du/dt is the downward acceleration.

When the drag force balances the weight of the sphere there is no acceleration, so du/dt=0, and equation (2) can be rearranged to be:

V_{S}(ρ_{S}−ρ_{F})g−F_{D}=0 (3)

and finally,

F_{D}=V_{S}(ρ_{S}−ρ_{F})g (4)

However the drag force is F_{D}=f_{F}u_{T}.

### Where:

u_{T} is the velocity of sedimentation, and f_{F} is the friction factor.

Thus equation (3) can be further arranged to be:

$${u}_{T} = \frac{{V}_{S} ( {ρ}_{S} - {ρ}_{F} ) g}{{f}_{F}} \qquad ( 5 )$$

The centrifugal force generated in the vessel assembly 1 rotating at a constant speed is given by equation (6).

F_{C}=mω²r (6)

### Where

m is the mass of the vessel assembly,
ω is the speed of rotation given in rad/sec, and
r is the distance of the vessel assembly, from the axis of rotation.

It is possible to substitute the acceleration of gravity in equation (4) by the artificial acceleration generated in a centrifuge ω²r, and equation (4) rearranges to be:

$${u}_{T} \frac{d r}{d t} = \frac{{V}_{S} ( {ρ}_{S} - {ρ}_{F} ) {ω}^{2} r}{{f}_{F}} \qquad ( 7 )$$

The rate at which particles sediment is given by the Svedberg equation or Sedimentation coefficient, s, and is defined as the ratio of the terminal velocity to the driving force acting on it per unit mass (the centrifugal force), or

$$s = \frac{d r / d t}{{ω}^{2} r} = \frac{{V}_{S} ( {ρ}_{S} - {ρ}_{F} )}{{f}_{F}} \qquad ( 8 )$$

but in general, since V_{S}=m_{S}/ρ_{S},

$$s = \frac{d r / d t}{{ω}^{2} r} = \frac{{m}_{S} ( {ρ}_{S} - {ρ}_{F} )}{{ρ}_{S} {f}_{F}} = \frac{{m}_{S}}{{f}_{F}} ( 1 - \frac{{ρ}_{F}}{{ρ}_{S}} ) \qquad ( 9 )$$

The frictional coefficient f_{F} is related to the size and shape of the particle. For a sphere of radius r_{S}, equation (7) becomes (10)

f_{F,sphere}=6πrμ (10)

### Where:

μ is the viscosity of the medium.

Then the sedimentation coefficient for a sphere can be found to be:

$${s}_{spehere} = \frac{d r / d t}{{ω}^{2} r} = \frac{2 {r}_{S}^{2} ( {ρ}_{S} - {ρ}_{F} )}{9 μ} \qquad ( 11 )$$

The unit of sedimentation is conveniently defined as the Svedberg, S, equivalent to 10⁻¹³ sec, since the sedimentation coefficient for most of the biological macromolecules is 10⁻¹³ sec. The sedimentation coefficient for particles is sometimes found empirically and can be related back to equation (7) to obtain the sedimentation velocity dr/dt=sω²r. For example, erythrocytes, where S has been found to be 10⁵S, will settle at unit gravity (dr/dt=sg) at a rate of approximately 1 mm/hr (or 0.3 μm/s). However, this rate scales in a centrifuge by the square of the angular speed but also does proportionally to the radial position of the cell. This means that for an angular velocity of 100 rad/s and a radius of 5 cm, the sedimentation velocity would increase fifty-fold to a value of 50 mm/hr (or 14 μm/s).

In addition to analysis based on physical separation of the sample to its constituents so as to identify for example somatic cell percentages or the proportion of fat within a sample volume, the teaching of the present invention can be extended in certain arrangements to enzymatic analysis of the milk sample either concurrently or sequentially with the centrifugal separation of the milk sample to its constituents. For example using a vessel such as that described heretofore, it is also possible to provide a method for diagnosis of mastitis using the enzyme N-acetyl-ss-D-glucosaminidase (hereinafter called NAGase). It is known in the art that that mastitis in cows may be diagnosed using the enzyme NAGase. Such analysis has however heretofore required separate testing. The vessel assembly 1 described heretofore may be included in a reaction system which allows a farmer to detect mastitis locally on a farm without the need to send milk samples to an off site testing facility. In such an arrangement, the vessel assembly 1 may be used for simultaneously analysing milk using both physical and enzymatic analytic techniques. NAGase assays will be appreciated as being exemplary of the type of enzymatic assays that may be integrated with the vessels 4 and arranged to be in fluid communication with the milk sample charged to the vessels 4. NAGase present in the milk sample, with the use of suitable colorimetric or fluorometric labels or dyes, causes a colour or fluorescence change and as a result is readily detectable using a colorimetric or fluorometric analysis. It will also be understood that NAGase is exemplary of an enzymatic assay and that other assays such as LDH (Lactate Dehydrogenase) which is also a marker of mastitis, similar to NAGase, could also be employed.

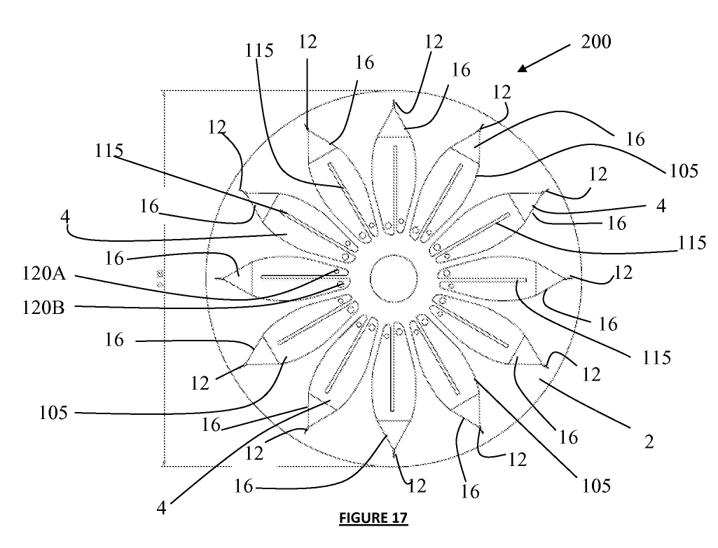

To provide a detection of this colour change, a photodection arrangement as illustrated in FIG. 16 may be used. In an exemplary arrangement, the photodetection arrangement may include the blue laser 64 of the disc drive unit 22 which excites the liquid sample. The laser 64 of the disc drive unit 22 causes the samples in the vessels 4 of the vessel assembly 1 to fluoresce. A photodetector 65 located on the same side of the disc 2 with respect to the laser 64 is operable for carrying out a colorimetric comparison between the milk sample and a control colour scheme for determining if the milk has mastitis. The photodetector 65 may employ the optics of the disc drive unit 22 and may itself be part of the optical system of the drive unit 22. It will be appreciated that in this arrangement conventional optical components within a DVD player are usefully employed in both excitation of a sample and subsequent colorimetric or fluorometric analysis on such an excited sample.

It will be appreciated that the vessel arrangement described heretofore provides an apparatus for counting and measuring cells. In this way it may be considered a cytometer. To provide for the necessary counting, a counter may be used in combination with the vessel for counting the somatic cells in the blind channel. Program logic in the form of executable software or the like may also be employed to estimate the fat content of the sample by measuring area/length of the fat band in the body portion 10. To provide an at-point of measurement display, digital displays may be employed to provide digital readings of the counted cells and the estimated fat content resultant from the measurement process. The digital display of the disc drive unit 22 may be used to display the quantified results. Thus, a person with no or little scientific skills such as a farmer may conduct the analysis of a milk sample to determine if a cow has mastitis and/or the fat content thereof locally on the farm.

The present invention also relates to a technique for measuring lipids in milk by spectrophotometry. A lipid test in this context is based on the property that fatty acids absorb light proportional to their concentration. A substance is added to the milk which precipitates proteins and hydrophobic peptides that interfere with blue measurement. Blue light from the laser 64 of the disc drive unit 22 may be used to excite the milk samples within the vessels 4 of the vessel assembly 1. The level of absorption of blue light by the milk samples or the emission of fluorescence as a result of excitation by the blue light is measured by a photodetector 65 for determining the concentration of lipids.

It will be appreciated that it is further possible to use the milk analysis apparatus for effecting a determination of the protein content within a particular milk sample. In a manner similar to that described with reference to the enzymatic analysis it will be appreciated that an analysis apparatus such as that provided in accordance with the present teaching could be used in effecting a determination of the concentration of protein in the sample, as determined for example by a colorimetric assay for protein. It will be understood that the presence or otherwise of protein in a milk sample is an indicator of the quality of the milk and not necessarily of mastitis and in this context it will be understood that an apparatus such as that provided in accordance with the present teaching should not be construed as being limited to a mastitis detector.

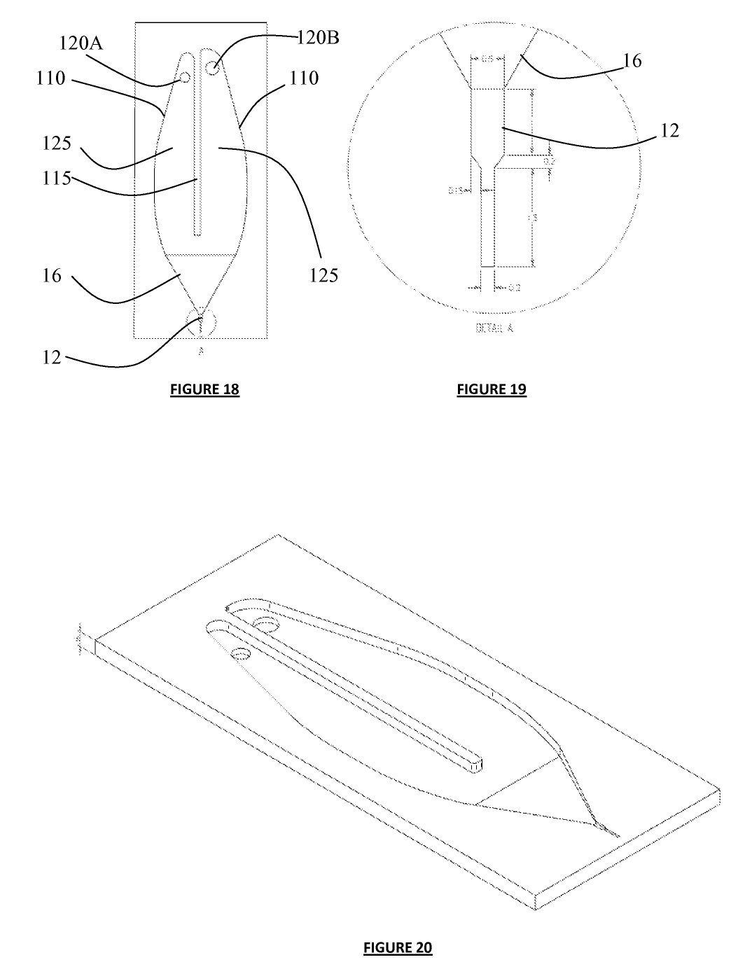

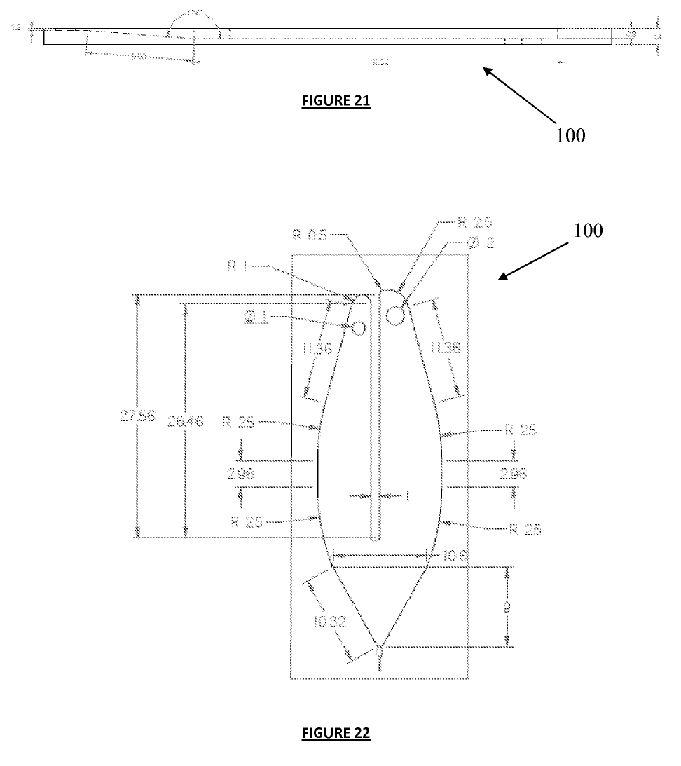

Referring now to FIGS. 17 to 22 which illustrates another vessel assembly 100 which is substantially similar to the vessel assembly 1, and like components are indicated with the same reference numerals. The main difference between the assembly 100 and the assembly 1 is the shape of the vessels 4. The V-shape channel 16 and the trap 12 in assemblies 1 and 100 are substantially identical.

The elongated body portion 10 of assembly 1 is replaced with a curved shaped arcuate body portion 105 in assembly 100. The curved shape portion 105 comprises a pair of spaced apart convex shaped side walls 110, which bulge outwardly from the inner volume of each vessel. The convex shape of the side walls 110 allows more vessels 4 to be located on the disc 2. In this exemplary arrangement twelve vessels are formed on a disc 2 with a foot print corresponding to that of a standard CD. An elongated central rib 115 is provided in each curved portion 105 which increases rigidity and the structural integrity of the vessels 4. The central rib 115 of each vessel 4 extends radially from the axis of rotation of the disc 2/disc drive unit 22 such that the ribs 115 define corresponding spokes on the disc 2.

It will be appreciated that the introduction of any fluid to a sealed or blind container requires a simultaneous discharge of the displaced gas that is within the container. In this way, when milk is being charged to the vessels 4 gas (air) may sometimes get trapped in body portion 10 which would limit the amount of milk which could be loaded to the vessels 4, or delay its introduction requiring for example agitation of the vessel to displace the trapped gas. In this arrangement, first and second inlets are provided within the vessel, the second inlet allowing for the escape of any air within the volume of the vessel upon introduction of a fluid into the first inlet. The provision of these first and second inlets desirably requires a modification of the body portion 105 of the vessel so as to facilitate this expulsion of the air that was previously present in the vessel. The inlet to the curved portion 105 is provided in the form of two apertures 120A and 1208 located on respective opposite sides of the central rib 115. The apertures 120A and 1208 are desirably offset with respect to each other. The central rib 115 divides the curved portion 110 into two regions 125 on respective opposite sides thereof. The apertures 120A and 120B safe guard against airlocks occurring while milk is being charged to the curved portion 105. Milk is loaded to the vessel 100 through the aperture 120A while the aperture 1208 provides an outlet for gas. In this way any gas present in the vessel will be biased downwardly through the introduction of the milk towards the end of the rib. At this juncture the path of least resistance is along the other side of the rib and out the aperture 120B.

It will be understood that what has been described herein are exemplary embodiments of milk analysis apparatus. While the present invention has been described with reference to exemplary arrangements it will be understood that it is not intended to limit the teaching of the present invention to such arrangements as modifications can be made without departing from the spirit and scope of the present invention. It will be understood by those skilled in the art that the apparatus of the present invention may be configured to enable the measurement of the quantity of biological cells or other particles in a liquid sample by their separation from the liquid using centrifugation to cause sedimentation and localization of the cells or particles at the distal end, relative to an axis of rotation, of a tube, channel, or chamber that is oriented substantially perpendicular to the axis of rotation of a platform or substrate upon or within which said tube, channel, or chamber (“vessel”) is located, thereby facilitating the optical, electrical, or other analysis of the quantity of said cells or particles after their sedimentation and localization is substantially complete. The carrier member may include a plurality of vessels each including a narrowed, tapered, constricted trap, or substantially funnel-shaped region at the end distal from the axis of rotation and sized to accommodate the anticipated range of quantities of cells or particles to facilitate their measurement. The vessels enable the analysis of cell or particle content of a multiplicity of liquid samples simultaneously or in time sequence. The vessels may be suitable for the measurement of one or more components of a liquid sample of lower density than the majority liquid by way of the centrifugal localization of said components into “bands” nearer the axis of rotation than the balance of the sample, as in the case of liquid fat or oil emulsified or suspended in an aqueous sample such as milk. The vessels may be suitable to support or contain one or more molecularly specific assays, measured by optical, electrical, or other means, for particular individual, groups, or classes of proteins, enzymes, nucleic acids, or other molecular biological species dissolved in said sample. The carrier member may be suitable to be rotated at angular velocities typical of the operation of compact disk (CD) or digital video disk (DVD) drives known in the art for data storage and retrieval applications and which are exemplary of a centrifuge which provides a means for rotating the carrier member to provide for centrifugation of a fluid sample within the sample vessel.

It will be appreciated from the foregoing that a drive unit which may used for effecting a rotation of the sample volume can be provided by the drive unit of conventional CD or DVD player. These drive units should be considered within the context of the present teaching as centrifuges and are exemplary of the type of means for rotating the vessel that may be usefully employed. Use of the optical head that is provided as part of the CD or DVD player may also be used for analysis of a fluid sample. It will be appreciated that optical heads for use, in for example BluRay™ technology, provide multiple available and distinct wavelengths which could be individually used in an analyser as provided in the context of the present disclosure. For example BluRay red light could be employed in the analysis of somatic cells as part of the physical analysis and the available blue light could be used in excitation of the label or dye associated with NaGase for the enzymatic analysis.

While the teaching of the present specification has been referenced to exemplary arrangements which provide for the isolation of somatic cells to allow for mastitis testing, it will be appreciated that in addition to the isolation of such somatic cells that an analysis apparatus as provided by the present teaching may be usefully employed in characterisation of the quality of the milk by combining two or more different tests using the same fluidic platform and on the same sample of milk. Such tests have been described with reference to one or more exemplary tests selected from: (1) an analysis of the quantity of cream or fat in the milk sample, determined by centrifugal separation of the cream or fat; (2) the determination of the concentration of protein in the sample, determined by a colorimetric assay for protein; (3) the determination of the concentration of the enzyme NAGase in the sample, determined by colorimetric or fluorometric assay using appropriate antibodies and indicative colorimetric or fluorometric labeling methods. Using an analysis apparatus as provided in accordance with the present teaching it is possible to combine the output from two or more individual tests that are performed on the same milk sample, either concurrently or in sequential steps, to provide an overall output which is based on a combination of the individual tests. Such a characterisation of the milk quality based on combination of two or more individual tests to provide overall output from the analysis apparatus may be provided in the form of a visual indicator to the user to enable them to determine at the point of testing an indication of the milk quality. It will be appreciated that in effecting a combination of the outputs from the individual tests, that the outputs from the individual tests may be weighted dependent on their contribution to the overall output from the apparatus. It will be further appreciated that where two or more tests are provided on the same originating milk sample that the output of each of the tests could also be individually provided to the user who may for example wish to ascertain the specifics of the results of any one of the individual tests that was conducted.

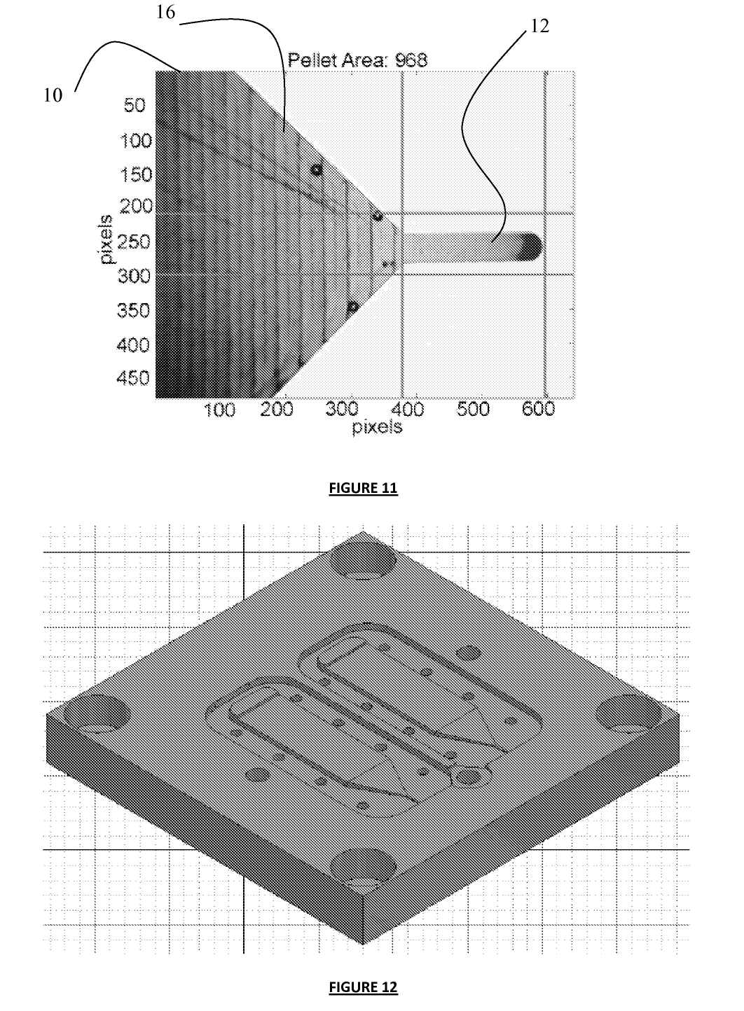

It will be further understood that while the vessels have been described as being integrated with the disc, it will be readily apparent to those skilled in the art that the vessels could be provided independently of the disc and may be attached to the disc by a suitable securing means. Additionally, it will be appreciated that the vessel assembly could be formed by using moulding techniques, or other mass fabrication techniques rather than milling. The mould of FIG. 12 may be for example be used for moulding the vessels of the vessel assembly 1, and the mould of FIG. 20 may be used for moulding the vessels of the vessel assembly 100. In this way it will be understood that the invention is to be limited only insofar as is deemed necessary in the light of the appended claims.

Similarly the words comprises/comprising when used in the specification are used to specify the presence of stated features, integers, steps or components but do not preclude the presence or addition of one or more additional features, integers, steps, components or groups thereof.

1.-105. (canceled)

106. A milk analysis apparatus for detecting mastitis in a milk sample by isolating somatic cells suspended therein using centrifugal sedimentation to form them into a pellet, the apparatus comprising:
a rotatable carrier member, the carrier member comprising a plurality of individual vessels, each vessel configured to hold an individual milk sample, each vessel comprising:
a body portion, an inlet to facilitate charging milk into the body portion and a trap to capture a somatic cellular pellet comprised of somatic cells suspended in the milk sample, and
wherein operable rotation of the carrier member biases the somatic cells within the milk sample towards the respective trap of each vessel.

107. A milk analysis apparatus as claimed in claim 106, wherein the carrier member has an axis of rotation, the inlet of each vessel located proximal to the axis of rotation and the trap of each vessel located distal to the axis of rotation.

108. A milk analysis apparatus as claimed in claim 106, wherein the trap of each vessel is formed by a constricted portion of the vessel, the somatic cellular pellet being optically identifiable within the trap.

109. A milk analysis apparatus as claimed in claim 106, wherein the trap of each vessel is elongated, the vessel terminating with the trap.

110. A milk analysis apparatus as claimed in claim 107, wherein the body portion of each vessel is formed by one of:
an elongated region of the vessel;
an arcuate region of the vessel, and
the body portion is located intermediate the trap and the axis of rotation.

111. A milk analysis apparatus as claimed in claim 106, wherein a transverse cross sectional area of the body portion of each vessel is substantially greater than a transverse cross sectional area of the respective trap such that the body portion defines a major volume of the vessel and the trap defines a minor volume of the vessel.

112. A milk analysis apparatus as claimed in claim 106, wherein each vessel further comprises a guide portion to guide the cells into the trap, the guide portion located intermediate the body portion and the trap and wherein the guide portion tapers inwardly from the body portion to the trap.

113. A milk analysis apparatus as claimed in claim 106, wherein the carrier member forms a disc, and the body portion of each vessel comprises a pair of spaced apart major surfaces with a pair of spaced apart minor surfaces extending therebetween.

114. A milk analysis apparatus as claimed in claim 113, wherein the inlet is formed on one of the major surfaces such that milk may be introduced into the vessel in a direction substantially parallel with an axis of rotation of the vessel.

115. A milk analysis apparatus as claimed in claim 106, wherein the trap is dimensioned to accommodate up to a predetermined amount of cells therein.

116. A milk analysis apparatus as claimed in claim 106, configured such that identification of a pellet of at least a particular size within a trap is operably used to ascertain the presence of mastitis within the milk sample.

117. A milk analysis apparatus as claimed in claim 106, wherein the inlet comprises a first aperture and a second aperture to facilitate charging milk into the body portion and to allow air previously present in the vessel to exit the vessel.

118. A milk analysis apparatus as claimed in claim 117, wherein the body portion includes a pair of spaced apart curved side walls.

119. A milk analysis apparatus as claimed in claim 117, the vessel further comprising an elongated rib that extends radially from an axis of rotation of the vessel, the rib dividing the body portion into discrete regions on respective opposite sides thereof and wherein the pair of apertures are located on respective opposite sides of the rib.

120. A milk analysis apparatus as claimed in claim 106, further comprising at least one of:
an enzymatic assay for facilitating enzymatic analysis;
a protein assay for facilitating an analysis of protein levels within the milk sample;
quantifying means for quantifying the fat content of the milk sample subsequent to rotation of the vessel; and
one or more molecularly specific assays for particular individual, groups, or classes of proteins, enzymes, nucleic acids, or other molecular biological species dissolved in said sample

121. A milk analysis apparatus as claimed in claim 106, wherein the carrier member allows optical transmission of light therethrough to facilitate optical analysis of the milk samples.

122. A milk analysis system comprising:
a rotatable carrier member, the carrier member comprising a plurality of individual vessels, each vessel configured to hold an individual milk sample, each vessel comprising:
a body portion, an inlet to facilitate charging milk into the body portion and a trap to capture a somatic cellular pellet comprised of somatic cells suspended in the milk sample, and
wherein operable rotation of the carrier member biases the somatic cells within the milk sample towards the respective trap of each vessel; and
an analysis apparatus configured to provide an analysis of one or more of somatic cell level, fat content, protein level and/or enzymatic levels within the milk sample, the system optionally configured to provide an output indicative of the quality of the milk by combining analysis related to the somatic cell level with one or more of fat content, protein level and/or enzymatic levels within the milk sample to provide an overall output of the milk quality.

123. A milk analysis system as claimed in claim 122, wherein the analysis apparatus comprises:
a processor configured to couple outputs from a physical analysis of the milk sample with at least one molecular analysis of the milk sample to provide an overall output indicative of the quality of the milk sample; and
one or more optical heads communicatively coupled to the processor and positioned to capture optical information from the vessels.

124. A milk analysis system as claimed in claim 117, further comprising:
a rotatable drive having a spindle unit coupled to rotate the carrier member, the carrier member having an opening sized to accommodate the spindle of the rotatable drive unit therein to facilitate rotation of the carrier member.
